(19) United States
(12) Patent Application Publication
(10) Pub. No.: US 2008/0242633 A1
(43) Pub. Date: Oct. 2, 2008

(54) Methods of modulating apoptosis and platelet production using variants of cytochrome c

(76) Inventors: Elisabeth Cramer, Paris (FR); Serge Fichelson, Lagny-sur-Marne (FR); Ian Malcom Morison, Dunedin (NZ)

Correspondence Address: BORSON LAW GROUP, PC, 1320 WILLOW PASS ROAD, SUITE 490, CONCORD, CA, 94520-5232, US

(21) Appl. No.: 12/070,599

(22) Filed: Feb. 20, 2008

Related U.S. Application Data
(63) Continuation of application No. PCT/NZ2006/000200, filed on Aug. 4, 2006.

(30) Foreign Application Priority Data
Aug. 5, 2005 (NZ) 541,666

(51) Int. Cl. A61K 31/70; A01N 1/02

(52) U.S. Cl. 514/44; 435/2

(57) ABSTRACT

The invention relates to a method for using the gene or protein containing CYCS(Gly42-Ser) to enhance cellular apoptosis. The method can be used to modulate apoptosis to treat conditions associated with an abnormal rate of apoptosis, in particular to treat conditions associated with increased cell growth, for example hyperplasia, hypertrophy, cancer, neoplasia or the like. The invention also relates to the use of the gene or protein containing CYCS(Gly42-Ser) for stimulating platelet release from megakaryocytes, and also to the treatment of thrombocytopenia using the platelets.

### FIELD OF INVENTION

This invention relates to the use of a variant of human cytochrome c. Specifically, this invention relates to the involvement of a mutant of cytochrome c in the modulation of apoptosis and the stimulation of platelet release from megakaryocytes.

### BACKGROUND OF INVENTION

Apoptosis, also known as programmed cell death, describes an orderly process through which excess and old cells die. Apoptosis occurs during development to limit the number of cells within the nervous system, kidneys, thymus, spleen, and other organs. Several pathways contribute to apoptosis. A key component of apoptosis in some cells is the release of cytochrome c from mitochondria. After release into the surrounding cytosol, cytochrome c activates one of the apoptosis pathways. Cytochrome c binds to Apaf-1, and recruits caspase-9, to form the apoptosome that triggers the downstream activation of the effector mechanisms of the cell death pathways. Many cancers have developed mechanisms to decrease or block apoptosis, and there is intense scientific interest in the development of drugs that will enhance apoptosis in cancer cells.

Cytochrome c protein is highly conserved in all eukaryotes, with 20 internal amino acids (including glycine 42) being invariant across 113 species studied (Banci L., et al., J. Biol. Inorg. Chem., 1999, 4:824-37). Cytochrome c is located in the mitochondria of all aerobic cells. It is involved in the electron transport system of the oxidative phosphorylation pathway. It accepts electrons from cytochrome B and transfers them to cytochrome oxidase. In addition to its role in oxidative phosphorylation, release of cytochrome c from the mitochondrial intermembrane space results in nuclear apoptosis. Binding of Apaf-1 to cytochrome c allows Apaf-1 to form a ternary complex with, and activate, the initiator procaspase-9 in the presence of dATP. Active caspase-9 then activates downstream effector caspases, beginning the death cascade.

The cellular mechanisms underlying some human diseases, such as some cancers and autoimmune disease, include mechanisms that prevent or decrease apoptosis, thereby enhancing survival of cells that would normally die by programmed cell death (see for example Brunner T. and Mueller C. Essays in Biochemistry, 2003, 39:119). There is considerable interest in novel therapies that might modulate apoptotic activity (see for example Fischer U. and Schultze-Osthoff K., Pharmacological Reviews, 2005, 57:187-215, and Reed J. C. and Pellecchia M., Blood, 2005, 106:408-18).

Platelets, also known as thrombocytes, are small circulating fragments of bone marrow cells termed megakaryocytes. Megakaryocytes are large cells that develop and mature in the bone marrow. When mature, the megakaryocyte's cytoplasm demarcates into finger-like processes known as proplatelets that subsequently fragment into hundreds of platelets. Fragmentation of proplatelets into platelets occurs in the bone marrow sinusoids or in the circulation, but does not normally occur in the bone marrow space itself. Demarcation of the megakaryocyte cytoplasm into proplatelets, uses a mechanism that is very similar to that used for the orderly cell death, known as programmed cell death or apoptosis (de Botton S., et al. Blood, 2002, 100(4):1310-7). The regional activation of apoptosis involves the release of cytochrome c from mitochondria, and subsequently the activation of caspase 3. Work reported by Clarke et al. confirmed the role of activation of apoptosis in platelet formation (Clarke M. C. H., et al., J. Cell Biol., 2003, 160(4):577-587). The role of apoptosis in proplatelet production is also strongly supported by the observation that platelet production is impaired by overexpression of the anti-apoptotic protein BclxL in megakaryocytes (Kaluzhny Y., et al., Blood, 2002, 100(5):1670-8).

Platelets are involved in formation of blood clots and wound healing. Thrombocytopenia is a medical condition characterized by decreased numbers of platelets in the blood. Implications of this condition can vary from a relatively minor form in which blood clotting is somewhat slower than normal to more severe forms, in which thrombocytopenia is a life-threatening condition.

In some instances it has been discovered that thrombocytopenia is genetically associated. In one instance (PCT/US2004/003786, published as WO 2004/072256) a family having mild thrombocytopenia, with platelet counts in the range of about 70−150×10⁹ platelets/1 were found to have a mutation in cytochrome c.

In patients having severe thrombocytopenia, the usual therapy is infusion of platelets derived from donors. However, due to transfusion of blood products, patients can become inadvertently infected with infections, including bacterial and viral organisms. It is important to prevent transmission of blood-borne viral diseases, such as human immune deficiency virus (HIV), hepatitis A, hepatitis C, and other infectious agents, through non-infectious therapies. Production of platelets in culture would provide a way of enhancing the safety of platelet transfusions.

Human megakaryocytes can be induced to produce large numbers of proplatelet extensions and small numbers of platelets are released into the culture media (Cramer E. M., et al., Blood, 1997, 89:2336-2346; Choi E. S., et al., Blood, 1995, 85:403-413). Some cell lines, such as Meg-01, show megakaryocytic features, and can be induced to form platelets in some experimental conditions. For example, transfection of Meg-01 cells with a scinderin containing vector, led to release of platelet like particles (Zunino R., et al., Blood, 2001, 98:2210-2219). Exposure of Meg-01 cells to S-nitrosoglutathione led to a marked production of functional platelet-sized particles having morphological features specific for platelet forms (Battinelli E., et al., Proc. Natl, Acad, Sci., USA, 2001, 98:14458-14463). However, to date, it has not been possible to produce platelets in culture suitable for medical applications.

### SUMMARY OF INVENTION

This invention describes new methods for enhancing apoptosis.

It has been discovered that a functional mutation in the protein cytochrome c, known to cause a condition characterized by mild thrombocytopenia, has surprisingly been found to increase apoptosis of megakaryocytes.

The mutation in cytochrome c in the affected members of a family results in the substitution of the glycine at position 42 by a serine residue. The amino acid position is based on the NCBI Reference Sequence for cytochrome c (NP_{—}061820.1 GI:11128019). This mutation is herein termed CYCS(Gly42-Ser). The sequence of the coding DNA for this abnormal cytochrome c is provided.

This invention includes methods for using the gene or protein containing CYCS(Gly42-Ser) to enhance cellular apoptosis. The methods can be used to modulate apoptosis to treat conditions associated with an abnormal rate of apoptosis, in particular to treat conditions associated with increased cell growth, for example hyperplasia, hypertrophy, cancer, neoplasia or the like.

The invention particularly provides a method for modulating apoptosis in a cell comprising administering to said cell any one of:
(a) an isolated oligonucleotide comprising SEQ ID NO: 1;
(b) a compliment of (a);
(c) an expression vector comprising a promoter operably linked to a sequence of (a) or (b); or
(d) an isolated peptide having the sequence of SEQ ID NO:4.

The invention also includes methods for using the gene or protein containing CYCS(Gly42-Ser) to stimulate platelet release from megakaryocytes. The method can also be performed in vitro for the production of platelets, and includes platelets produced by the methods, and their use in the treatment of disorders associated with platelet production.

The invention particularly provides a method for stimulating platelet release from megakaryocytes comprising administering to said megakaryocyte any one of:
(a) an isolated oligonucleotide comprising SEQ ID NO: 1;
(b) a compliment of (a);
(c) an expression vector comprising a promoter operably linked to a sequence of (a) or (b); or
(d) an isolated peptide having the sequence of SEQ ID NO:4.

The invention also particularly provides a method for the in vivo stimulation of platelet release from megakaryocytes comprising the steps of:
(i) administering to said megakaryocyte any one of:
   (a) an isolated oligonucleotide comprising SEQ ID NO: 1;
   (b) a compliment of (a);
   (c) an expression vector comprising a promoter operably linked to a sequence of (a) or (b); or
   (d) an isolated peptide having the sequence of SEQ ID NO:4;
(ii) culturing the megakaryocytes under conditions suitable for platelet release.

### BRIEF DESCRIPTION OF FIGURES

The invention is described with respect to particular embodiments thereof. Other features and aspects of the invention are presented in the Figures, in which:

FIG. 1: depicts the in vivo phagocytosis of platelets by bone marrow macrophages in an affected family member, a consequence of abnormal release of platelets into the bone marrow space rather than into bone marrow sinusoids. Bone marrow biopsy from the family member shows that platelet production occurs in the marrow rather than the bone marrow sinusoids as normal. The platelet (A) does not reach the circulation and, as a consequence, is engulfed by the macrophage (B). 4450× magnification

FIG. 2: depicts the formation of platelets from peripheral blood hematopoietic stem (CD34+) cell-derived megakaryocytes grown in vitro. Numerous platelets (A) have been produced in the culture dish. (Day 11 of culture). 1950× magnification.

FIG. 3: shows the mass spectrometry spectra of expressed mutant CYCS(Gly42-Ser) (A) and wildtype cytochrome c (B). The molecular weight of wildtype cytochrome c is measured at 12 232 Da, which is in close agreement with the calculated value of 12 233.9 Da. CYCS(Gly42-Ser) has a measured mass of 12 265 Da. The difference in size between the mutant and wild-type forms of 33 Da is in close agreement with the expected difference of 30 Da.

FIG. 4: shows the enhanced caspase 3 activation that is induced by CYCS(Gly42-Ser) in a cell-free assay. Cytochrome c at concentrations of 7.5 and 10.0 nM was added to cytosol containing 1 mM dATP and 50 μM Ac-DEVD-AMC. The rate of production of AMC is a measure of caspase-3 activation. Symbols and corresponding cytochrome c concentrations: ▪ and □: 10.0 nM. ♦ and ⋄: 7.5 nM. The AMC production rate at a CYCS(Gly42-Ser) concentration of 7.5 nM (⋄) is higher than that of wild type cytochrome c at 10.0 nM (▪).

### DETAILED DESCRIPTION

The present invention is based on the surprising discovery that the mutation CYCS(Gly42-Ser) in cytochrome c induces apoptosis. In certain embodiments the invention provides for a method for modulating apoptosis in a cell using CYCS(Gly42-Ser) (SEQ ID NO: 4). The method may involve the administration to a cell an isolated oligonucleotide comprising the polynucleotide for CYCS(Gly42-Ser) or a compliment thereof.

The sequence could be administered in the form of an expression vector comprising a promoter operably linked to a sequence encoding CYCS(Gly42-Ser). In such a method the expression vector could be introduced into a cell using known techniques and expression of the sequence would modulate apoptosis of the cell and/or surrounding cells. In certain embodiments an inducible promoter could be used to control the expression of CYCS(Gly42-Ser).

Alternatively the cell could be treated with the CYCS(Gly42-Ser) peptide (SEQ ID NO:4). The method may involve treating the cell under conditions that allow CYCS(Gly42-Ser) to enter the cell.

The method can be used as a treatment of a condition associated with an abnormal rate of apoptosis, for example a condition associated with increased cell growth. Such conditions, could include, but are not limited to hyperplasia, hypertrophy, cancer, or neoplasia or the like.

Accordingly the invention also provides for a pharmaceutical composition comprising CYCS(Gly42-Ser) or a gene encoding CYCS(Gly42-Ser), or an expression vector comprising a promoter operably linked to a sequence encoding CYCS(Gly42-Ser). The CYCS(Gly42-Ser) or a gene encoding CYCS(Gly42-Ser), or an expression vector comprising a promoter operably linked to a sequence encoding CYCS(Gly42-Ser), may be formulated with other components, as known in the art, suitable for administration to modulate apoptosis in the cell.

In further aspects the invention also provides for the use of any one of: (a) an isolated oligonucleotide encoding CYCS(Gly42-Ser); (b) a compliment of (a); (c) an expression vector comprising a promoter operably linked to a sequence of (a) or (b); or (d) an isolated peptide having the sequence of SEQ ID NO:4; in the production of a composition for modulating apoptosis in a cell.

The medicament may be suitable for the treatment of a condition associated with an abnormal rate of apoptosis, and may include a condition associated with increased cell growth. Such conditions may include, but are not limited to, hyperplasia, hypertrophy, cancer, or neoplasia, or the like.

In a still further aspects, this invention provides for a method for stimulating platelet release from megakaryocytes using CYCS(Gly42-Ser). The method may involve the administration to a megakaryocyte an isolated oligonucleotide comprising the polynucleotide for CYCS(Gly42-Ser) or a compliment thereof.

The sequence may be administered in the form of an expression vector comprising a promoter operably linked to a sequence encoding CYCS(Gly42-Ser). In such a method the expression vector could be introduced into a cell using known techniques and expression of the sequence would stimulate platelet release from the megakaryocyte and/or surrounding megakaryocytes. In certain embodiments an inducible promoter could be used to control the expression of CYCS(Gly42-Ser).

Alternatively, the megakaryocyte could be treated with the CYCS(Gly42-Ser) peptide (SEQ ID NO:4). The method may involve treating the megakaryocyte under conditions that allow CYCS(Gly42-Ser) to enter the cell.

A yet further aspect of the invention is a method for the in vivo stimulation of platelet release from megakaryocytes. The method may comprise the step of administering to said megakaryocyte a sequence encoding CYCS(Gly42-Ser) or a compliment thereof.

The sequence may be administered in the form of an expression vector comprising a promoter operably linked to a sequence encoding CYCS(Gly42-Ser). In such a method the expression vector could be introduced into a cell using known techniques and expression of the sequence would stimulate platelet release from the megakaryocyte and/or surrounding megakaryocytes. In certain embodiments an inducible promoter could be used to control the expression of CYCS(Gly42-Ser).

Alternatively, the megakaryocyte could be treated with the CYCS(Gly42-Ser) peptide (SEQ ID NO:4). The method may involve treating the megakaryocyte under conditions that allow CYCS(Gly42-Ser) to enter the cell.

The megakaryocytes may be derived from any cell line that can be induced to form a megakaryocyte. The cell line may include, but is not limited to, any one of peripheral blood or bone marrow; stem cells obtained from peripheral blood or bone marrow; a megakaryocyte cell line, or the like.

The method also includes the step of culturing the cell under suitable conditions for platelet production. This may include culturing the megakaryocyte with suitable growth factors to optimize platelet production.

In a further step the platelet may be purified by any suitable technique and allows for the production of platelets in vivo. The in vitro production of platelets offers a new way of producing platelets in an efficient and sterile environment for use in the treatment of an animal in need of such treatment, in particular an animal having thrombocytopenia. The method also provides a unique and useful too for the study of platelet production.

### Familial Thrombocytopenia

A family with inherited mild thrombocytopenia (low platelet count) was previously discovered. Their platelet counts varied from 73 to 148×10⁹/L. Twenty six related persons with thrombocytopenia were identified. The low platelet count is inherited in an autosomal dominant pattern. The family members had no obvious symptoms, except perhaps a mild tendency to easy bruising and nose bleeds. This is a form of thrombocytopenia that has not been identified and published previously. Thrombocytopenia was identified in 26 family members, and the DNA of 25 affected family members examined. The affected family members were otherwise well. No other inherited traits were identified. In particular, the family members appear to have a normal life expectancy, they do not have an increased rate of degenerative diseases of the nervous system, they do not have disorders of the immune system, and they have a below average rate of cancer.

### Identification of the Causative Mutation

Using DNA from 25 affected family members and 38 unaffected family members and 7 spouses, a genetic linkage of the thrombocytopenia trait to the short arm of chromosome 7 was established. A mutation at nucleotide position 132 of exon 2 of the human cytochrome c gene that was present in all members of the family who had inherited thrombocytopenia (low platelets) was then described. The mutation is present in a heterozygous state, i.e. the affected persons have one mutant copy of the cytochrome c gene and one normal copy.

The DNA sequence of the mutant protein-coding region of human cytochrome c from affected members of the family is as follows (as previously described in PCT/US2004/003786):

SEQ ID NO:1

ATGGGTGATGTTGAGAAAGGGAAGAAGATTTTTATTATGAAGTGTTCCCA
GTGCCACACCGTTGAAAAGGGAGGCAAGCACAAGACTGGGCCAAATCTCC
ATGGTCTCTTTGGGCGGAAGACAAGTCAGGCCCCTGGATACTCTTACACA
GCCGCCAATAAGAACAAAGGCATCATCTGGGGAGAGGATACACTGATGGA
GTATTTGGAGAATCCCAAGAAGTACATCCCTGGAACAAAAATGATCTTTG
TCGGCATTAAGAAGAAGGAAGAAAGGGCAGACTTAATAGCTTTATCTCAA
AAAAGCTACTAATGAGTAA

The normal coding region of human cytochrome c is as follows:

SEQ ID NO:2

ATGGGTGATGTTGAGAAAGGCAAGAAGATTTTTATTATGAAGTGTTCCCA
GTGCCACACCGTTGAAAAGGGAGGCAAGCACAAGACTGGGCCAAATCTCC
ATGGTCTCTTTGGGCGGAAGACAGGTCAGGCCCCTGGATACTCTTACACA
GCCGCCAATAAGAACAAAGGCATCATCTGGGGAGAGGATACACTGATGGA
GTATTTGGAGAATCCCAAGAAGTACATCCCTGGAACAAAAATGATCTTTG
TCGGCATTAAGAAGAAGGAAGAAAGGGCAGACTTAATAGCTTATCTCAAA
AAAGCTACTAATGAGTAA

The mutation in the nucleotide sequence above results in the substitution of the glycine at position 42 by a serine residue. The glycine at position 42 is a highly conserved amino acid being invariant in 113 studied eukaryote species (Banci L., et al., J. Biol. Inorg. Chem., 1999; 4:824-37).

The NCBI Reference Sequence (NP_{—}061820 cytochrome c [Homo sapiens] gi|11128019|ref|NP_{—}061820.1|[11128019]) provides the following amino acid sequence for human cytochrome c:

SEQ ID NO:3

MGDVEKGKKIFIMKCSQCHTVEKGGKHKTGPNLHGLFGRKTGQAPGYSYT
AANKNKGIIWGEDTLMEYLENPKKYIPGTKMIFVGIKKKEERADLIAYLK
KATNE

The sequence of the mutant cytochrome c variant in the family is predicted based on the genetic code:

SEQ ID NO:4

MGDVEKGKKIFIMKCSQCHTVEKGGKHKTGPNLHGLFGRKTSQAPGYSYT
AANKNKGIIWGEDTLMEYLENPKKYIPGTKMIFVGIKKKEERADLIAYLK
KATNE

Using allele-specific oligonucleotide PCR the mutation was detected in 24 affected family members, whereas none of 27 non-affected family members and none of 319 non-family individuals showed the mutation. The mutation is not present in any of the RNA and EST sequences for cytochrome c in the Unigene section of GenBank, and thus represents a novel cytochrome c sequence. Unigene contains 2057 cytochrome c sequences of which approximately 950 include exon 2 sequences flanking the site of the mutation.

### The CYCS(Gly42-Ser) Mutation Alters Platelet Release In Vivo and In Vitro

Abnormal release of platelets from megakaryocytes of affected family members in vivo and in vitro has been observed.

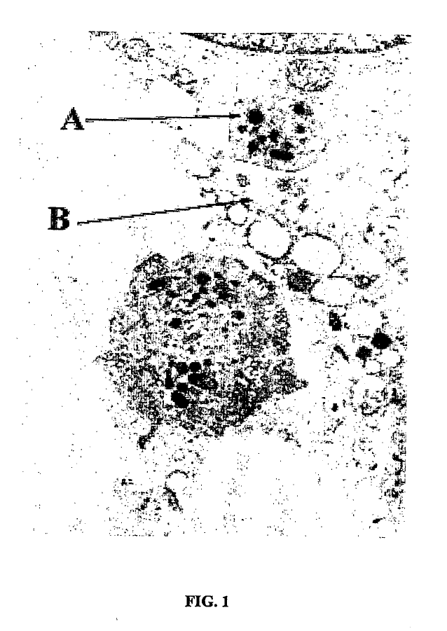

Evidence for altered in vivo platelet release: Transmission electronmicroscopy of femoral bone marrow from an affected family member showed platelets within the bone marrow space. Femoral head bone fragments were dissected from the femoral head of a 76 year old female member of the family who carries the CYCS(Gly42-Ser) mutation. The bone fragments were fixed in glutaraldehyde in 0.1 M cacodylate buffer, processed for conventional transmission electronmicroscopy and photographed. Examination of the electronmicrographs clearly showed the presence of platelets within the marrow space and within bone marrow macrophages (FIG. 1). Release of platelets directly into the bone marrow space was abnormal and was only observed in pathological states. In addition, the nuclei of the megakaryocytes, within the bone marrow space, showed increased apoptotic changes as assessed by electronmicroscopy.

The observation of platelets within the marrow space means that the CYCS(Gly42-Ser) mutation causes abnormal platelet production. Because platelet production occurs by regional apoptosis within the megakaryocyte cytoplasm, CYCS(Gly42-Ser) mutation causes enhanced release of platelets within the bone marrow space rather than into the circulation. Released platelets are then destroyed by phagocytosis by macrophages. Furthermore, CYCS(Gly42-Ser) mutation shows a role in inducing apoptosis.

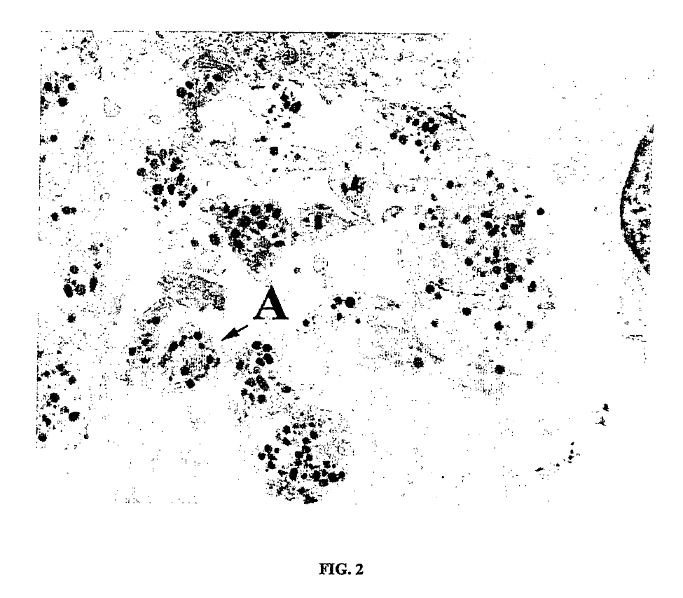

Evidence for altered in vitro platelet release: Blood-derived stem cells from an affected family member were induced to form megakaryocytes in culture. After 6 days of culture, large numbers of platelets were release into the media. This phenomenon was initially interpreted as bacterial contamination of the culture, but later confirmed, by electronmicroscopy (FIG. 2), to be platelet release. Peripheral blood CD34+ cell-derived megakaryocytes from normal individuals release small numbers of platelets after 7-11 days in culture (Cramer E M et al, Blood 1997; 89:2336-2346, Choi E S et al, Blood 1995; 85:403-413). The observation of release of large numbers of platelets after only 6 days of culture is very abnormal and indicates that the CYCS(Gly42-Ser) mutation is associated with premature or inappropriate platelet release in these culture conditions.

Specifically, peripheral blood from an affected 24 yr old member of the family was obtained, and CD34+ blood cells were isolated from the peripheral blood. Mononuclear cells were separated on a ficoll-hypaque gradient (Lymphoprep; Nycomed Pharma, Oslo, Norway), washed, and then used for isolation of CD34+ cells by cell sorting on a FACS Vantage flow cytometer (Becton Dickinson). CD34+ cells were cultured in Iscove's modified Dulbecco medium (IDDM; GIBCO, Paisley, UK) with penicillin/streptomycin/glutamine and 11.5 μmol/L α-thioglycerol (Sigma) supplemented with polyethylene glycol (PEG)-rHuMGDF (Amgen Corp., Thousand Oaks, Calif.) at a final concentration of 10 ng/mL. Cultures were performed in serum free conditions at 37° C. in a fully-humidified atmosphere containing 5% CO₂ in air (Alimardani G. et al., Thrombosis and Haemostasis, 2002, 88:1039-46). Other publication describing megakaryocyte culture include Cramer E. M., et al., Blood, 1997, 89:2336-2346 and Choi E. S., et al, Blood, 1995, 85:403-413).

### Expression and Purification of CYCS(Gly42-Ser)

Full length CYCS(Gly42-Ser) was inserted into a pcDNA3 vector (pcDNA3::HCS-A). The mutation was introduced into the vector pBTR (Human Cc) (Amp^{r}). pBTR (Human Cc) had previously been converted from pBTR (hCc), an expression vector for horse cytochrome c and yeast heme lyase (heme lyase is required for the expression of cytochrome c in bacteria). The conversion involved site-directed mutagenesis of the horse cytochrome c coding sequence to convert the amino acid sequence to that of wildtype human (Olteanu A, et al, Biochem Biophys Res Commun. 2003; 312(3):733-40). pBTR (Human Cc) was then modified so that the vector expressed CYCS(Gly42-Ser). A BglII-XhoI fragment in pcDNA::HCS-A was amplified by polymerase chain reaction (PCR) and the amplicon was cloned onto pBTR (Human Cc) backbone produced from digestion with the BglII and XhoI. The resulting plasmid encodes human CYCS(Gly42-Ser).

pBTR containing either CYCS(Gly42-Ser) or wildtype CYCS sequence was transfected into Escherichia coli strain BL21(DE3). The expressed cytochrome c was purified as previously described (Olteanu A., et al., Biochem. and Biophys. Res. Commun., 2003, 312:733-740). The protein was purified from dialysate using a 5.0 mL HiTRAP SP Sepharose column (Amersham Biosciences). All procedures were performed at room temperature at a flow rate of 2.0 mL/min. The column was equilibrated with 5.0 mL of low salt buffer (1.76 g/L NaH₂PO₄, 7.31 g/L Na₂HPO₄, pH 7.3). Supernatant was applied to the column and proteins were elute in a linear, 10-column volume (50 mL) gradient from low to high salt buffer (0.652 g/L NaH₂PO₄, 4.10 g/L Na₂HPO₄ and 58.4 g/L NaCl, pH 6.9). Fractions (2 mL) were collected and purity of the fractions was assessed by A₄₁₀/A₂₈₀ measurement and SDS-polyacrylamide gel electrophoresis. Concentrations of cytochrome c were calculated from absorbance at A₄₁₀ using an extinction coefficient of 10.61 mM⁻¹mm⁻¹.

### The Structure CYCS(Gly42-Ser)

Spectrophotometry: The characteristic red color together with the absorbance spectrum of purified CYCS(Gly42-Ser) indicated that haem had been bound to apo-cytochrome c to form the functional protein (holo-cytochrome c). That is, the structure was at least relatively unaffected by the mutation. By spectrophotometry the absorbance spectra of wild type cytochrome c and CYCS(Gly42-Ser) were not noticeably different.

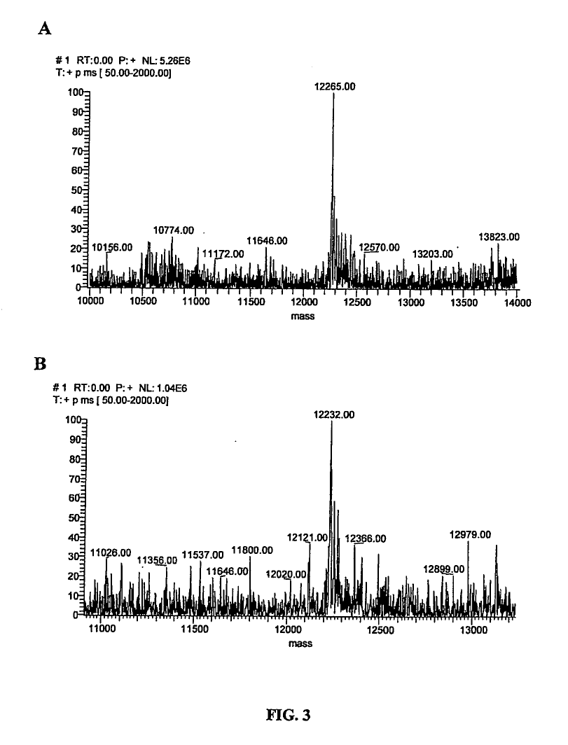

Mass spectrometry: The molecular weight of wild type cytochrome c was measured (FIG. 3) as 12 232 Da, which is in close agreement with the calculated value of 12 233.9 Da (Jeng W. Y., et al., J. Bioenergetics and Biomembranes, 2002, 34:423-31). CYCS(Gly42-Ser) had a measured molecular weight of 12 265 Da (FIG. 3). As glycine and serine have a molecular weight of 75 and 105 Da respectively, the difference in molecular weight between wild type cytochrome c and CYCS(Gly42-Ser) is consistent with a glycine to serine substitution.

### Pro-Apoptotic Function of CYCS(Gly42-Ser)

The apoptotic function of CYCS(Gly42-Ser) was tested in a cell free caspase-3 activation assay. One of the normal functions of cytochrome c is to bind to Apaf-1 to form the apoptosome, along with caspase-9. Activation of caspase 9 results in activation of caspase 3. The ability of wild type cytochrome c and CYCS(Gly42-Ser) to activate this caspase cascade was measured by cleavage of Ac-DEVD-AMC, a substrate of caspase 3, into its fluorescent product AMC. To do this cytochrome c was introduced into a reaction mixture containing cytosolic extract from cultured U-937 cells, together with some Ac-DEVD-AMC.

In detail, U-937 cells were grown in RPMI-1640 media with 10% (v/v) heat-inactivated fetal bovine serum, 50 unit/mL penicillin G and 50 mg/mL streptomycin. Cultures were incubated at 37° C. with 5.0% CO₂ in humidified air. Prior to extraction of cytosol, cells were adjusted to a density of 0.5×10⁶ cell/mL and grown in fresh media for 20 hours. Cells were centrifuged at 400 g for 5 min at 4° C. and washed once in ice-cold PBS. The pellet was resuspended to a density of 80×10⁶ cell/mL in ice-cold extraction buffer. The extraction buffer contained 20 mM HEPES, pH adjusted to 7.5 with KOH; 10 mM KCl, 1.5 mM MgCl₂, 1 mM EGTA, 1 mM EDTA, fresh DTT and PMSF to a final concentration of 1 mM and 0.1 mM respectively and one protease inhibitor cocktail tablet (Complete Mini, EDTA-free tablet, from Roche, Basel, Switzerland) freshly dissolved in every 10 mL of lysis buffer. After fifteen-minute incubation in extraction buffer on ice, cells were lysed by passing through a 27 gauge needle ten times. The cell lysate was centrifuged at 1000 g for 10 min at 4° C. The supernatant was further centrifuged at 100 000 g at 4° C. for one hour. The resultant S-100 supernatant was snap frozen and stored at −80° C.

To assess the ability of cytochrome c to activate caspase 3, a 60 μL mixture containing 30 μL of cytosolic extract from cultured U-937 cells, 1 mM dATP (Amersham Biosciences, Buckinghamshire, England) and 50 μM Ac-DEVD-AMC (Calbiochem, EMD Biosciences, Inc. San Diego, Calif., USA) in assay buffer (100 mM HEPES, pH adjusted to 7.25 with NaOH; 10% (w/v) sucrose, 0.1% (w/v) CHAPS, DTT freshly added to 5.0 mM), was placed in each well of a black 96-well MicroWell™ plate (Nunc, Roskilde, Denmark). The wild type and mutant cytochrome c was diluted in assay buffer to the specified concentrations and added at time zero. Reactions were carried out at 37° C. Negative controls received the same volume of assay buffer. Fluorescence was measured as relative fluorescence unit (RFU) at an excitation wavelength of 390 nm and an emission wavelength of 490 nm by a POLARstar OPTIMA microplate reader (BMG Labtechnologies, Offenburg, Germany). Readings were taken at 1 minute intervals for 120 minutes. RFU was converted to ‘pmol of AMC produced’ using an AMC standard curve.

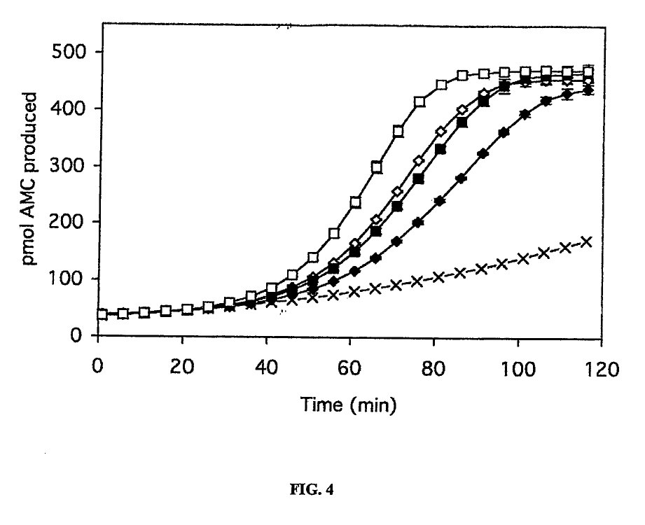

The AMC production rate by CYCS(Gly42-Ser) was always higher than that for wild type cytochrome c and the same concentration. For example, at a concentration of 7.5 nM, the CYCS(Gly42-Ser) had caspase-3 activation activity greater than that for wild type cytochrome c at 10 nM. That is, at a 7.5 nM concentration the CYCS(Gly42-Ser) had activity approximately 1.4 fold greater than wild type (see FIG. 4).

The applicant has surprisingly shown that CYCS(Gly42-Ser) shows enhanced caspase-3 activation in the cell free assay system, indicating pro-apoptotic properties. Cultured megakaryocytes carrying one mutant copy of CYCS(Gly42-Ser) showed enhanced platelet release in culture. Given that platelet formation is dependent on apoptosis, this observation is consistent with enhanced apoptosis.

Affected family members who carry one mutant copy of CYCS(Gly42-Ser) have thrombocytopenia that is attributable to dysfunctional release of platelets into the bone marrow space, consistent with pro-apoptotic activity in vivo.

Wherein in the foregoing description reference has been made to integers or components having known equivalents, such equivalents are herein incorporated as if individually set fourth.

Although the invention has been described by way of example and with reference to possible embodiments thereof, it is to be appreciated that improvements and/or modifications may be made without departing from the scope or the spirit thereof.

### INDUSTRIAL APPLICABILITY

The invention provides a method for using the gene or protein containing CYCS(Gly42-Ser) to enhance cellular apoptosis. The method can be used to modulate apoptosis to treat conditions associated with an abnormal rate of apoptosis, in particular to treat conditions associated with increased cell growth, for example hyperplasia, hypertrophy, cancer, neoplasia or the like. The invention also relates to the use of the gene or protein containing CYCS(Gly42-Ser) for stimulating platelet release from megakaryocytes, and also to the treatment of thrombocytopenia using the platelets.

1. A method for modulating apoptosis in a cell comprising administering to said cell any one of:
(a) an isolated oligonucleotide comprising SEQ ID NO: 1;
(b) a compliment of (a);
(c) an expression vector comprising a promoter operably linked to a sequence of (a) or (b); or
(d) an isolated peptide having the sequence of SEQ ID NO:4.

2. A method according to claim 1, for the treatment of a condition associated with an abnormal rate of apoptosis.

3. A method according to claim 1, for the treatment of a condition associated with increased cell growth.

4. A method according to claim 3, wherein the condition is hyperplasia, hypertrophy, cancer, or neoplasia.

5. A method according to claim 1, wherein the method is performed in vitro or in vivo.

6. The use of any one of:
(a) an isolated oligonucleotide comprising SEQ ID NO: 1;
(b) a compliment of (a);
(c) an expression vector comprising a promoter operably linked to a sequence of (a) or (b); or
(d) an isolated peptide having the sequence of SEQ ID NO:4;
in the production of a composition for modulating apoptosis in a cell.

7. The use according to claim 6, for the treatment of a condition associated with an abnormal rate of apoptosis.

8. The use according to claim 6, for the treatment of a condition associated with increased cell growth.

9. The use according to claim 6, wherein the condition is hyperplasia, hypertrophy, cancer, or neoplasia.

10. A method for stimulating platelet release from megakaryocytes comprising administering to said megakaryocyte any one of:
(a) an isolated oligonucleotide comprising SEQ ID NO: 1;
(b) a compliment of (a);
(c) an expression vector comprising a promoter operably linked to a sequence of (a) or (b); or
(d) an isolated peptide having the sequence of SEQ ID NO:4.

11. The method according to claim 10, wherein the method is preformed in vitro or in vivo.

12. The method according to claim 10, for the treatment of disorders of platelet production.

13. A method for the in vivo stimulation of platelet release from megakaryocytes comprising the steps of:
(i) administering to said megakaryocyte any one of:
(a) an isolated oligonucleotide comprising SEQ ID NO: 1;
(b) a compliment of (a);
(c) an expression vector comprising a promoter operably linked to a sequence of (a) or (b); or
(d) an isolated peptide having the sequence of SEQ ID NO:4;
(ii) culturing the megakaryocytes under conditions suitable for platelet release.

14. A method according to claim 13, wherein the megakaryocytes are derived from peripheral blood or bone marrow; stem cells obtained from peripheral blood or bone marrow, a megakaryocyte cell line, or a cell line that can be induced to form a megakaryocyte.

15. The method according to any claim 13, comprising a further step of isolating said platelets.

16. A platelet produced by the method of any one of claims 13 to 15.

17. The use of a platelet according to claim 16, for administration to an animal in need thereof.

18. The use according to claim 17, for the treatment of thrombocytopenia.


SEQUENCE LISTING (4 sequences)

SEQ ID NO: 1 - DNA, 318 bp, Homo Sapians
atgggtgatgttgagaaaggcaagaagatttttattatgaagtgttcccagtgccacaccgttgaaaagggaggcaagcacaagactgggccaaatctccatggtctctttgggcggaagacaagtcaggcccctggatactcttacacagccgccaataagaacaaaggcatcatctggggagaggatacactgatggagtatttggagaatcccaagaagtacatccctggaacaaaaatgatctttgtcggcattaagaagaaggaagaaagggcagacttaatagcttatctcaaaaaagctactaatgagtaa

SEQ ID NO: 2 - DNA, 318 bp, Homo Sapians
atgggtgatgttgagaaaggcaagaagatttttattatgaagtgttcccagtgccacaccgttgaaaagggaggcaagcacaagactgggccaaatctccatggtctctttgggcggaagacaggtcaggcccctggatactcttacacagccgccaataagaacaaaggcatcatctggggagaggatacactgatggagtatttggagaatcccaagaagtacatccctggaacaaaaatgatctttgtcggcattaagaagaaggaagaaagggcagacttaatagcttatctcaaaaaagctactaatgagtaa

SEQ ID NO: 3 - protein, 105 aa, Homo Sapians
MGDVEKGKKIFIMKCSQCHTVEKGGKHKTGPNLHGLFGRKTGQAPGYSYTAANKNKGIIWGEDTLMEYLENPKKYIPGTKMIFVGIKKKEERADLIAYLKKATNE

SEQ ID NO: 4 - protein, 105 aa, Homo Sapians
MGDVEKGKKIFIMKCSQCHTVEKGGKHKTGPNLHGLFGRKTSQAPGYSYTAANKNKGIIWGEDTLMEYLENPKKYIPGTKMIFVGIKKKEERADLIAYLKKATNE